(19) United States
(12) Patent Application Publication
(10) Pub. No.: US 2024/0033735 A1
(43) Pub. Date: Feb. 1, 2024

(54) Method Of Diagnosing Cardiovascular Diseases, And A Device For Separating Components Of A Fluid Sample For Diagnosing Cardiovascular Diseases

(71) Applicant: Hong Kong Centre for Cerebro-Cardiovascular Health Engineering Limited, Hong Kong (HK)

(72) Inventors: Bee Luan Khoo, Hong Kong (HK); Mingze Zhu, Hong Kong (HK)

(21) Appl. No.: 17/876,787

(22) Filed: Jul. 29, 2022

(51) Int. Cl. B01L 3/00; G01N 33/68

(52) U.S. Cl. CPC B01L 3/502753; G01N 33/6893; B01L 2300/0883; B01L 2300/0864; G01N 2800/32; B01L 2400/0487; B01L 2200/0652

(57) ABSTRACT

The present invention relates to a method of diagnosing cardiovascular disease (CVD). The method comprises obtaining a whole blood sample from a subject; separating components of the whole blood sample into a plurality of fluid fractions; collecting one or more selected fluid fractions comprising one or more separated components of the whole blood sample, wherein the one or more separated components comprise cardiac cells such as cardiomyocytes; and detecting expression of one or more cardiovascular disease-associated biomarkers from the cardiomyocytes from the selected fluid fractions thereby determining one or more related cardiovascular diseases in the subject.

### FIELD OF THE INVENTION

The invention relates to the separating, focusing and/or detecting of components of a fluid sample obtained from a subject for the diagnosis of cardiovascular diseases (CVDs) of the subject. Particularly, the invention relates to using microfluidic devices to detect cardiovascular diseases (CVDs).

### BACKGROUND OF THE INVENTION

Cardiovascular diseases (CVDs) are one of the leading illnesses and causes of death worldwide. In 2019, there were an estimated 17.9 million cardiovascular diseases related deaths, accounting for about 32% of global mortality. CVD is a general term that refers to a wide range of abnormal conditions in the heart and/or blood vessels, such as but are not limited to heart diseases or failures, strokes, arrhythmia, and problems with heart valves. The risk factors for CVDs may include hypertension, smoking, physical inactivity, unhealthy diet, overweight, and/or rise in blood lipid and glucose levels, among which hypertension is associated with the highest risk for CVDs. With the COVID-19 pandemic, studies revealed that hospitalized COVID-19 patients with CVDs would have higher risks of death than those without CVDs, and the limitation of physical activity due to restriction would considerably increase the risk of CVD conditions and mortality. Moreover, the recurrence rate of CVDs is particularly high compared to other diseases, for example, up to about 75% with myocardial infarction history. As such, diagnosis and prognosis of CVDs are vital to the global healthcare systems.

However, diagnosing and long-term monitoring of CVDs remain challenging to the current clinical and healthcare settings. This is because the development of most CVDs is generally asymptotic, and the detectable level of most clinically available biomarkers for CVDs diagnosis is generally low, especially at the early stage of CVD development, which may result in false-negative detection results. Very often, the clinicians may only be allowed a very short period of treatment window, and thus a slight delay in treatment may significantly increase the severity of the disease. Furthermore, the available technologies for fast detection of CVDs are known to be insufficient or lacking sensitivity and/or efficiency. The development of new techniques for more effective detections of CVDs is therefore desirable.

### OBJECTS OF THE INVENTION

An object of the present invention is to provide a method for detecting or diagnosing cardiovascular diseases (CVDs) or related diseases.

Another object of the present invention is to mitigate or obviate to some degree one or more problems associated with known diagnostic techniques for CVDs, or at least to provide a useful alternative.

The above objects are met by the combination of features of the main claims; the sub-claims disclose further advantageous embodiments of the invention.

One skilled in the art will derive from the following description of other objects of the invention. Therefore, the foregoing statements of the object are not exhaustive and serve merely to illustrate some of the many objects of the present invention.

### SUMMARY OF THE INVENTION

In a first main aspect, the invention provides a method of diagnosing cardiovascular disease (CVD). The method comprises the steps of obtaining a sample fluid from a subject; separating components of the sample fluid into a plurality of fluid fractions; collecting one or more selected fluid fractions comprising one or more separated components of the sample fluid, wherein the one or more separated components comprise cardiac cells; detecting one or more cardiovascular disease-associated biomarkers from the cardiac cells from the selected fluid fractions thereby determining one or more related cardiovascular diseases in the subject.

In a second main aspect, the invention provides a device for separating components of a sample fluid obtained from a subject for diagnosing cardiovascular disease (CVD) of the subject. The device comprises at least one fluid passageway connecting, at two distal ends, an inlet where the sample fluid is loaded and a plurality of outlets where a plurality of fluid fractions carrying separated components of the sample fluid are collected; wherein the separated components collected at one or more selected fluid fractions comprise cardiac cells.

In a third main aspect, the invention provides a method of separating and focusing cardiac cells from a whole blood sample obtained from a subject for diagnosing cardiovascular disease (CVD) of the subject. The method comprises the steps of treating the whole blood sample to prepare a sample fluid; introducing the sample fluid to the device according to the second main aspect; separating components of the sample fluid into a plurality of fluid fractions; and collecting one or more selected fluid fractions comprising one or more separated cell components from the sample fluid, wherein the one or more separated cell components comprise cardiac cells.

The summary of the invention does not necessarily disclose all the features essential for defining the invention; the invention may reside in a sub-combination of the disclosed features.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing and further features of the present invention will be apparent from the following description of preferred embodiments which are provided by way of example only in connection with the accompanying figure, of which:

FIG. 1A is a schematic diagram showing a spiral microfluidic device according to an embodiment of the present invention;

FIG. 1B is a schematic diagram showing an embodied workflow for separating components of a fluid sample using the device of FIG. 1A according to the present invention;

FIG. 2 shows a representative fluorescence image of cardiomyocytes stained with Troponin I;

FIG. 3 shows the representative immunofluorescence images of rat cardiomyocytes collected from each of the five outlets of the device as shown in FIG. 1A after the separation;

FIG. 4A shows the distribution of cells collected from the different outlets when all of the cells from the cardiomyocyte-spiked blood sample are considered;

FIG. 4B shows the distribution of cells collected from the different outlets when only cardiomyocytes (CM) from the cardiomyocyte-spiked blood sample are considered;

FIG. 5A is a color drawing showing images of the normalized fluorescence intensity from four types of biomarkers, namely, CRP, HSP 70, Troponin I and Troponin T;

FIG. 5B shows the fluorescence intensity based on the expression of the four biomarkers;

FIG. 6A are the representative immunofluorescence images in color showing positive cardiomyocytes cell line control samples, samples from a healthy individual, and patient samples using Troponin T and Troponin I as biomarkers;

FIG. 6B shows the fluorescence signal count of patient samples for Troponin T;

FIG. 6C shows the fluorescence signal count of patient samples for Troponin I;

FIG. 6D shows the fluorescence signal count of the patient samples for Troponin T from target and non-target outlets; and

FIG. 6E shows the fluorescence signal count of the patient samples for Troponin I from target and non-target outlets.

### DESCRIPTION OF PREFERRED EMBODIMENTS

The following description is of preferred embodiments by example only and without limitation to the combination of features necessary for carrying the invention into effect.

Reference in this specification to “one embodiment” or “an embodiment” means that a particular feature, structure, or characteristic described in connection with the embodiment is included in at least one embodiment of the invention. The appearance of the phrase “in one embodiment” in various specifications does not necessarily refer to the same embodiment, nor are separate or alternative embodiments mutually exclusive of other embodiments. Moreover, various features are described, which may be exhibited by some embodiments and not by others. Similarly, various requirements are described, which may be requirements for some embodiments but not other embodiments.

Currently, the major causes of cardiovascular diseases (CVDs), which are believed to associate with lesions of blood vessels, are generally asymptotic, making the earlier detection of CVDs difficult. Common CVDs diagnostic techniques may include electrocardiograph (ECG), echocardiogram, blood biopsy, cardiac catheterization, chest x-ray, cardiac computed tomography (CT), and magnetic resonance imaging (MRI). However, these methods generally require sophisticated instruments and professional personnel, which makes them not easily adaptable in any clinical environment. Also, chest x-ray, CT, and cardiac catheterization involve radiation, and the latter is considered invasive. Therefore, the available methods may not be suitable or desirable for long-term monitoring and prognosis.

In contrast, blood biopsy may offer a non-invasive and relatively low-cost approach for CVDs detection by analyzing circulating biomarkers from a small amount of drawn blood from a subject. Therefore, it is considered more suitable for regular CVDs diagnosis and prognosis with a potential for point-of-care applications. Although some CVD biomarkers have already been applied to clinical diagnosis, many new biomarkers showing high clinical value to CVDs detection are still in the research phase (see Table 1).

TABLE 1
A summary showing a number of clinically available or potential biomarkers
for CVDs diagnosis (HSP70 = Heat shock protein 70; CRP =
C-reactive protein; FDP = Fibrin degradation product; miR = microRNA).

| | | | | Normal |
|---|---|---|---|---|
| | Relevant CVD | | | plasma |
| Biomarkers | conditions | Physiologic function | Origin | level |
| Cholesterol | Atherosclerosis | Production of steroid | All body's | <2 |
| | | hormones, vitamin D and | cells | g/L |
| | | bile | | |
| Fibrinogen | Intravascular | Formation of blood clots | Liver | 2-4 |
| | coagulation | upon bleeding | | g/L |
| HSP70 | Myocardial | Promotion of protein | Myocardium | <0.5 |
| | infarction | folding and intracellular | | ng/ml |
| | coronary artery | homeostasis | | |
| | disease | | | |
| CRP | Vascular | Removal of foreign | Liver | 0-3 |
| | inflammation | pathogens and damaged | | mg/L |
| | coronary artery | cells | | |
| | disease | | | |
| FDP | Myocardial | A substance that remains in | Blood clot | <10 |
| | infarction | the bloodstream after a | | μg/ml |
| | | blood clot dissolved | | |
| Troponin I | Obstructive | Transduction of calcium | Myocardium | 0-14 |
| and | coronary | signals to regulate muscle | | ng/ml |
| Troponin T | atherosclerosis | contraction | | |
| | Myocardial | | | |
| | infarction | | | |
| Fetuin-A | Coronary | Regulation of insulin | Liver | <0.4 |
| | atherosclerosis | signaling osseous | | mg/ml |
| | | calcification | | |
| miR-133a | Unstable | Cardiogenesis and | Myocardium | |
| | angina injured | regulation of cardiac action | | |
| | myocardium | potential | | |
| | myocardial | | | |
| | infarction | | | |
| miR-146a | Promotion of | Regulation of acute and | Central | |
| | angiogenesis | chronic inflammatory | nervous | |
| | myocardial | response | system | |
| | infarction | | | |
| Circulating | Aortic stenosis | Promotion of myocardial | Bone | Very low |
| stem cell | | and vascular homoeostasis | marrow | level |
| | | and regeneration | | |

Among various techniques used for blood biopsy, ELISA is one popular immunoassay for detecting proteins which shows good sensitivity and specificity. Nonetheless, it is often time-consuming as it generally requires two to three different antibodies for the test, leading to high costs. Another popular assay for detecting proteins is immunoturbidimetry which can be processed in minutes and, therefore, provides faster detections than ELISA. However, immunoturbidimetry requires a high limit of detection, for example, about 5 to 10 μg/ml; therefore, it may not apply to many biomarkers presented in low concentrations in the collected sample. Overall, most current immunoassays have limitations and require laboratory and/or sophisticated equipment, making regular disease management and prognosis difficult (see Table 2).

TABLE 2
The types of immunoassays commonly used for liquid biopsy.

| | Processing | Limit of | | | Instrument |
|---|---|---|---|---|---|
| Assay type | time | detection | | Cost | requirements |
| ELISA | 5-10 h | 0.2 | ug/ml | High | Medium |
| Immunoturbidimetry | Minutes | 5-10 | ug/ml | Medium | Low |
| Nephelometry | Minutes | 1 | ug/ml | Medium | High |

Recently, inertial microfluidics with spiral channels have gained popularity in research for applications such as rare cell detection for liquid biopsy and water purification for environmental study. Since antibodies are not involved, the isolated samples by microfluid platforms are highly viable, and their phenotypes can remain intact. The working principle of the spiral channeled microfluidic device attributes to the balance of an inertial lift force (F_{L}) and a Dean drag force (F_{D}) (Gou, Y, et al., Progress of Inertial Microfluidics in Principle and Application. Sensors (Basel), 2018. 18(6)). The F_{L} is caused by the balance of shear-gradient and wall-induced forces to allow particles in the fluid to migrate across the streamlines in a laminar flow. In addition to the F_{L}, migrating particles in a spiral channel will also experience a F_{D}. The F_{D} is caused by a centrifugal pressure gradient in the radial direction to constantly drag the particles circulating across the cross-section of the channel, which is also known as the Dean vortices (Zhang, J., et al., Fundamentals and applications of inertial microfluidics: a review. Lab on a Chip, 2016. 16(1): p. 10-34). When the particles are dragged at the inner channel location, the F_{L} and the F_{D} act in opposite directions to align the particles (Bhagat, A. A., et al., Inertial microfluidics for sheath-less high-throughput flow cytometry. Biomed Microdevices, 2010. 12(2): p. 187-95). Since the degree of F_{L} experienced by the particles is dependent on the size and deformability of the particles, particles which are large or stiff enough will experience appreciable F_{L} which balances the F_{D} to prevent the particles from moving along with the Dean vortices (Martel, J. M. and M. Toner, Inertial focusing in microfluidics. Annu Rev Biomed Eng, 2014. 16: p. 371-96; Guzniczak, E., et al., Deformability-induced lift force in spiral microchannels for cell separation. Lab on a Chip, 2020. 20(3): p. 614-625). Utilization of this phenomenon leads to size and deformability-based separation of the particle components of the fluid, which is simple, rapid, inexpensive and sensitive.

The present invention provides a simple, rapid and sensitive detection method for detecting or diagnosing CVDs which is applicable even in the early and acute stages of CVDs. The invention further provides a relatively low-cost and convenient method for long-term monitoring of conditions and statuses for patients with CVDs. Particularly, the invention applies a novel spiral microfluidic device, which separates, isolates, focuses and/or enriches cardiac cells or cardiac-associated cells from a patient's liquid biopsy sample, such as but is not limited to a whole blood sample. The separating and focusing of the cardiac cells from the particle components of the blood sample are due to the inertial migration of the components along the spiral channel or passageway of the microfluidic device, with the separation being dependent on physical properties such as differences in size and/or stiffness or deformability of the components. Unlike the traditional methods, the present invention allows fast detection of CVDs, with the test results generally achievable within two hours with high detection sensitivity. Therefore, the present invention is applicable to point-of-care diagnosis by using cardiac cells as cell-based biomarkers for diagnosing CVDs. With the present invention, the processing time in diagnosing the patient's sample for CVDs could be significantly shortened, and the long-term monitoring of conditions and statuses of the CVDs patients would become feasible.

In one embodiment, the present invention relates to a method of diagnosing cardiovascular disease (CVD). The method comprises the steps of obtaining a sample fluid from a subject, which can be a human and/or an animal subject. Preferably, the sample fluid is selected from the group consisting of whole blood, plasma, serum, urine or a combination thereof. More preferably, the sample fluid can be a whole blood sample drawn from the subject.

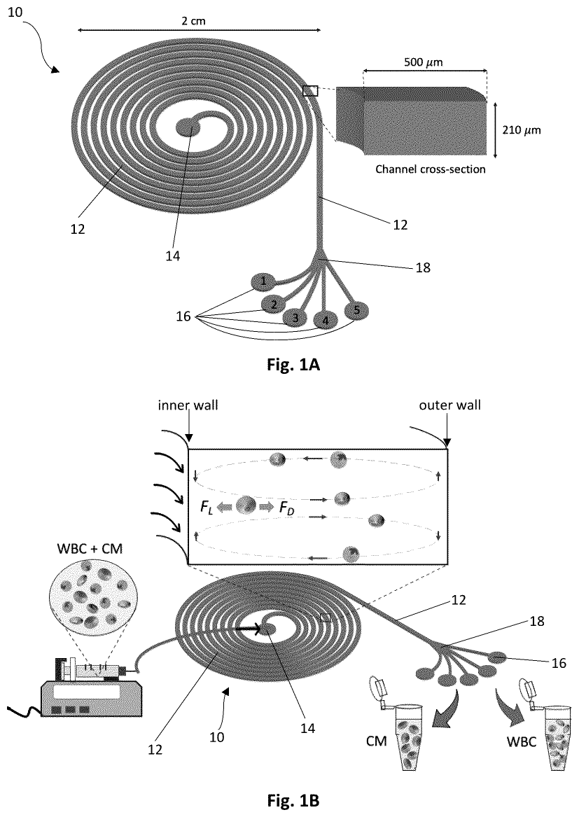

Preferably, the obtained whole blood sample from the subject is then treated for the subsequent separation. For example, the blood sample is first treated by lysing any blood cells such as red blood cells in the collected sample to reduce, minimize or avoid the distraction of the detection signal by the red blood cells. The treated sample will then be loaded, by any means, such as by way of syringe injection into a custom-made spiral microfluidic device for separating components of the treated sample. Particularly, the components will be separated, focused and collected as a plurality of fluid fractions. Referring to FIG. 1A, shown is an embodied spiral microfluidic device 10 according to the present invention. In this embodiment, the microfluidic device 10 comprises at least a curvilinear fluid passageway 12 connecting at its two distal ends, at least one inlet 14 where the sample fluid is loaded, and a plurality of outlets 16 where the corresponding plurality of fluid fractions are collected. Preferably, the curvilinear fluid passageway 12 of the microfluidic device 10 may comprise a spirally arranged channel forming about two to fifteen turns of substantially concentric spiral loops and, more preferably, 10 spiral loops as shown in the figure. In one embodiment, the curvilinear fluid passageway 12 forming ten substantially concentric spiral loops can be configured in a disc shape having a diameter of about 1 to about 5 cm, and preferably, about 2 cm. The inlet 14 is preferably arranged at a center of the substantially concentric spiral loops. In one further embodiment, the microfluid device 10 can be configured with a widened channel portion 18 connecting the curvilinear fluid passageway 12 and the plurality of outlets 16. The widened portion facilitates the separation of target cells from non-target cells by widening the distance between focused streamlines, allowing improved purity. Preferably, device 10 may comprise about two to ten outlets 16, such as five outlets as shown in the figure in the form of five sub-channels extending from the widened channel portion 18, for example.

In one embodiment, the curvilinear fluid passageway 12 may comprise a substantially rectangular cross-section, such as with a cross-sectional dimension of about 500 μm in width and about 200 μm in height, and preferably, about 210 μm in height. The plurality of outlets 16 may each preferably comprise a cross-sectional dimension of about 300 μm in width, about 200 μm in height, and preferably, about 210 μm in height. The sample fluid is arranged to pass along the spiral fluid passageway 12 at a flow rate preferably of about 1 ml/min to about 2 ml/min, and more preferably, about 1.7 ml/min.

The step of separating components of the sample fluid into a plurality of fluid fractions comprises separating and focusing the particle components of the sample fluid based on one or more properties selected from a group consisting but not limited to size, mass, shape, surface charges, density and deformability of the components. The separation and focusing of the particle components of the sample fluid into a plurality of fluid fractions is based on inertial migration of the components, as described earlier. Particularly, due to the curvilinear geometry of the fluid passageway 12 of the device 10, particle components of the sample fluid, which may comprise red blood cells, platelets, white blood cells and other cells, in case of a whole blood sample, will be subject to a combination of an inertial lift force F_{L} and a Dean drag force F_{D} causing a lateral equilibrium at a position near the inner wall of the fluid passageway 12. Depending on the ratio of the lift and the Dean drag forces, components with different sizes and/or stiffness may take distinct equilibrium positions, resulting in separating and focusing of the components into individual component streams, which will then be collected into different, separated sample fractions. Since the ratio of the lift force F_{L} to the Dean drag force F_{D} depends on the size of the particles, particle components with different diameters or sizes may equilibrate at distinct positions resulting in a continuous separation of multi-sized components mixture. Larger particle components may equilibrate at a position closer to the inner wall of the spiral microchannel while smaller particle components migrate away from the inner wall.

One or more selected sample fractions comprising one or more separated, targeted components of the sample fluid will then be collected, and preferably, the targeted components comprise cardiac cells, such as but are not limited to cardiomyocytes, for example. In the context of the present invention, the reference to cardiac cells may also comprise cardiac-associated cells. It is to be noted that cardiomyocytes have never been reported as a blood-based biomarker for CVD. FIG. 1B further shows a schematic workflow for separating and focusing components from the treated blood sample. The treated fluid sample is preferably introduced to the inlet 14 of the spiral microfluidic device 10. It is arranged to pass along the fluid passageway 12 at an optimal flow rate of 1.7 ml/min, controllable by a digital syringe pump. When compared to white blood cells (WBC), cardiomyocytes (CM) may experience larger inertial lift force F_{L} due to their larger sizes, allowing them to be strong enough to minimize the effect of the Dean drag force F_{D}. As such, cardiomyocytes are focused and migrated near the inner wall of the fluid passageway 12, leading to higher recovery in the target outlets, such as outlets 3 and 4.

After collecting the selected fluid fractions, the cardiomyocytes will be detected for one or more cardiovascular disease-associated biomarkers and/or physical biomarkers to determine one or more related cardiovascular diseases in the subject. In one embodiment, the cardiomyocytes will be detected for expression of one or more cardiovascular disease-associated biomarkers and/or physical biomarkers to determine one or more related cardiovascular diseases in the subject. Preferably, immunofluorescence from one or more cardiovascular disease-associated biomarkers from the separated cell fractions is detected. In one embodiment, the cardiovascular disease-associated biomarkers can be selected from the group consisting of troponin I, troponin T, heat shock protein 27, 60, 70 (HSP 27, 60, 70), C-reactive protein (CRP), cholesterol, fibrinogen, fibrin degradation product (FDP), fetuin-A, microRNA-133a, microRNA-146a, myoglobin, CK-MB, BNP & NT-proBNP, D-dimer, circulating stem cells, or a combination thereof. However, it is appreciated by the skilled person in the relevant art that the present invention shall not be limited by these specific examples of biomarkers. Instead, the present invention shall also encompass any known, clinically available and/or potentially applicable biomarkers considered possible or suitable for CVDs diagnosis.

In another aspect of the present invention, it provides a device 10, such as a spiral microfluid device 10, for separating particle components of a sample fluid obtained from a subject, such as a liquid biopsy sample, for diagnosing a cardiovascular disease (CVD) of the subject. The device 10 may comprise at least one fluid passageway 12, which connects, at its two distal ends, at least one inlet 14 where the sample fluid is loaded, and a plurality of outlets 16 where a plurality of fluid fractions comprising separated, target particle components of the sample fluid, are collected. Preferably, the separated, target particle components may comprise cardiac cells such as but are not limited to, cardiomyocytes.

In one embodiment, at least one fluid passageway 12 is preferably spirally arranged. As shown in the figures, it may form about two to fifteen turns of substantially concentric spiral loops, more preferably, ten turns of spiral loops. The inlet 14 is preferably arranged at a center of the substantially concentric spiral loops. The fluid passageway 12, prior to its dividing into a plurality of outlets 16, may further comprise a widened channel portion 18, which connects the fluid passageway 12 and the plurality of outlets 16. The widened portion facilitates the separation of target cells from non-target cells by widening the distance between focused streamlines, allowing improved purity. Preferably, the plurality of outlets comprises about two to ten outlets 16 branching off and extending from the widened channel portion 18. In one specific embodiment, the microchannel of the fluid passageway 12 may comprise a substantially rectangular cross-section having a dimension of about 500 μm in width and about 200 μm in height, and preferably, about 210 μm in height. Each of the plurality of outlets 16 may preferably have a cross-sectional dimension of about 300 μm in width and about 200 μm in height, and preferably, about 210 μm in height. The sample fluid is adapted to pass through the fluid passageway 12 at a flow rate of about 1 ml/min to about 2 ml/min, and preferably, about 1.7 ml/min.

In one further aspect of the present invention, it provides a method of separating and focusing cardiac cells from a whole blood sample obtained from a subject for diagnosing a cardiovascular disease (CVD) of the subject. The method comprises the steps of treating the whole blood sample to prepare a sample fluid, such as but not limited to lysing red blood cells from the whole blood sample to increase signal sensitivity of the subsequent detection process; introducing the treated sample fluid to the spiral microfluidic device 10 as described above; separating components such as particle components of the sample fluid into a plurality of fluid fractions; and then collecting one or more selected fluid fractions comprising one or more separated, target cell components from the sample fluid, wherein the one or more separated cell components comprise cardiac cells, such as cardiomyocytes. It is to be noted that cardiomyocytes have never been reported as a blood-based biomarker for CVD.

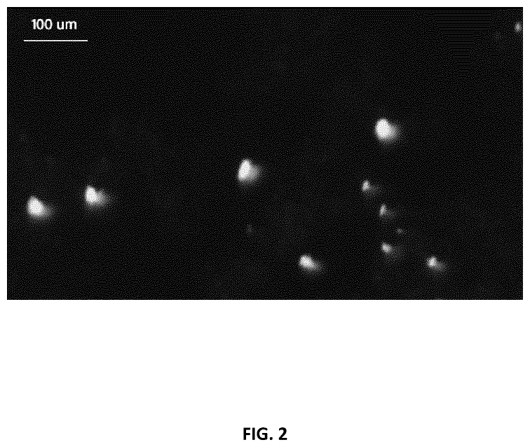

After separating the cardiac cells such as cardiomyocytes from the whole blood sample, the method may further comprise detecting expression of one or more cardiovascular disease-associated biomarkers from the cardiac cells obtained from the one or more selected fluid fractions. The step of detecting the expression of one or more cardiovascular disease-associated biomarkers from the cardiac cells may comprise detecting immunofluorescence from the one or more cardiovascular disease-associated biomarkers from the cardiac cells to determine or diagnose the associated CVDs of the subject. In one embodiment, the one or more cardiovascular disease-associated biomarkers may comprise any clinically available or potential CVD biomarkers, which can be, but are not limited to Troponin I, Troponin T, heat shock protein 27, 60, 70 (HSP 27, 60, 70), C-reactive protein (CRP), cholesterol, fibrinogen, fibrin degradation product (FDP), fetuin-A, microRNA-133a, microRNA-146a, myoglobin, CK-MB, BNP & NT-proBNP, D-dimer, circulating stem cells, or a combination thereof. In another embodiment, cardiomyocytes are heterogeneous in physical properties, including those with elongated shapes resembling cells under high shear. FIG. 2 illustrates a representative fluorescence image showing cardiomyocytes stained with Troponin I.

### EXAMPLE

### Methodology

### Cardiomyocytes Culture

SD rat cardiomyocytes were maintained in low glucose DMEM supplemented with 5% horse serum and 1% penicillin-streptomycin at 37° C. in a humidified environment with 5% CO₂. Cells were grown in 48 well plates, and the growth medium was changed every 48 hours. Cardiomyocytes were dissociated from the 48 well plates before being used for sorting. After removing the medium, the sample was washed with 1× phosphate buffer saline (1× PBS) three times, followed by adding 50 μL of 0.25% Trypsin-EDTA (ethylenediaminetetraacetic acid) for dissociation. After 5 minutes, the dissociation reaction was then neutralized with 200 μL of the medium, and cardiomyocytes were obtained by centrifugation at 1500 rpm for 5 minutes.

Lysis of Whole Blood

The whole blood sample was first mixed with red blood cell lysis buffer in 1:9 ratio under gentle agitation for 5 minutes and was centrifuged at 500 times gravity (×g) for 5 minutes to concentrate intact nucleated cells. The supernatant containing erythrocyte debris and plasma was removed, and the resulting nucleated cell pellet was immediately washed once with 1× PBS.

### Blood Sample Spiking

To prepare blood samples spiked with cardiomyocytes, 0.1 μL of Hoechst and 0.1 μL of Calcein AM were pipetted to a tube containing 100 μL cardiomyocytes solution, and the solution was placed in a 37° C. incubator for 30 minutes for staining. Then, the dye was washed with 1× PBS, and the sample was centrifugated. The nucleated cells were obtained by lysing 1 mL of whole blood and were concentrated at 100 μL. 0.1 μL of Hoechst was added to the cell solution and then placed in a 37° C. incubator for 30 minutes. The solution was washed with 1×PBS followed by centrifugation. The cardiomyocytes were then counted and added to the solution of nucleated cells from the whole blood lysis. The final spiked cell suspension was diluted to 3 mL with 1×PBS containing bovine serum albumin (BSA) at a final concentration of 0.5% BSA.

### Hoechst and Calcein Staining

The cardiomyocytes culture medium was removed, followed by washing with PBS. 0.5% Trypsin-EDTA buffer was used to detach cells from the cell culture dish. After all the cells were detached, the reaction was stopped by adding a complete cell culture medium. The cells were then stained with 5 μM Hoechst and 5 μM Calcein AM, respectively, and were incubated at 37° C. for 30 minutes.

### Spiral Microfluidic Device Fabrication

In one embodiment, the spiral microfluidic device 10 may consist of 10 spiral loops of fluid passageway 12 with a rectangular cross-section of about 500 μm of width×about 210 μm of height. The fluid passageway 12, which is arranged to spirally and outwardly extend from the centrally located inlet 14, forming 10 turns of spiral loops, will then linearly extend towards a widened main channel portion 18. The widened main channel portion 18 will then be divided into five sub-channels of outlets 16, each having a cross-sectional dimension of about 300 μm of width×about 210 μm of height (see FIG. 1A). The device 10 was preferably fabricated by standard soft lithography. Briefly, polydimethylsiloxane (PDMS) was prepared by mixing elastomer and curing agent in a ratio of 10:1 as specified by the manufacturer (Dow, Germany). The prepared PDMS was then poured onto an aluminum mold which was fabricated by micromachining. After degassing the PDMS in a vacuum pump, the PDMS on the mold was solidified by baking at 60° C. for 2 hours. The solidified PDMS was then taken off from the mold. The positions of the inlet 14 and outlets 16 were created by a biopsy puncher of 1.5 mm outer diameter (Integra, USA). Then, the PDMS was irreversibly bonded with a flat PDMS layer in a plasma cleaner. Finally, the bonded PDMS was baked at 60° C. for 30 minutes to strengthen the bonding.

### Device Processing

The spiral microfluidic device as formed was tested for leakage and flow consistency before usage. A sample resuspended in 2 ml 1× PBS was injected into a 5 ml syringe that was fluidly connected to the spiral microfluidic device via a plastic tubing (Tygon, USA). The sample was then introduced to the spiral device at an optimized flow rate of 1.7 ml/min, as controlled by a syringe pump (New Era Pump Systems Inc., USA). Output fraction from each outlet was then separately collected in a 1.5 ml centrifuge tube. The result will not be considered if the sample volumes collected at each of the five outlets were not consistent.

### Imaging and Analysis

Cells from each outlet will be collected for imaging under a fluorescent microscope. The cell suspension was transferred to an 18-well plate, and the cells were allowed to settle for 30 mins. Images for the whole well were taken. The captured images were processed by the ImageJ software (the U.S. National Institutes of Health). First, the

Hoechst signal in blue color was used to locate the cells. Next, the corresponding fluorescent intensity of the Calcein signal in green color was measured. Only a signal with an intensity greater than 25% of the maximum was considered a target of interest.

### Results

### Mechanisms of the Spiral Microfluidic Device

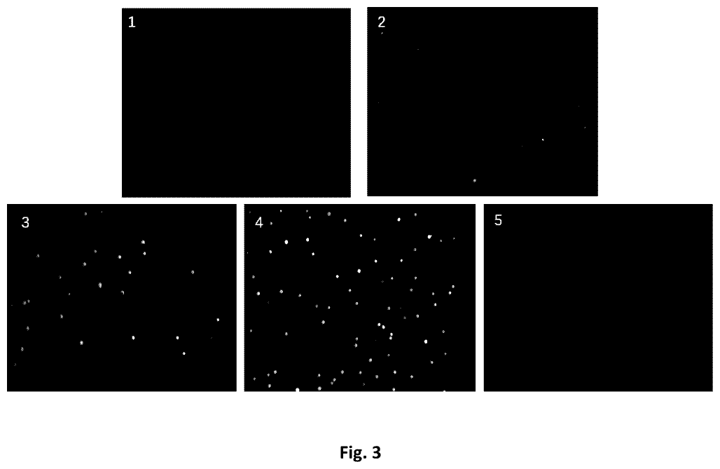

The embodied spiral microfluidic device of the present invention can be used to directly isolate cardio-related cells based on physical differences compared to blood cells. The fabrication of the device has been described in Wu, R., et al., Plasma heat shock protein 70 is associated with the onset of acute myocardial infarction and total occlusion in target vessels. Frontiers in Cardiovascular Medicine, 2021: p. 1153. Preferably, the device may comprise at least three major parts, namely, an inlet for sample input, a spiral region for cell focusing, and a plurality of outlets for separating cells into different fractions. In one embodiment, the cardiomyocytes were found to be concentrated in outlets 3 and 4, with the majority identified in outlet 4 (see FIG. 3). Briefly, while the cells were flowing along the spiral channel of the device, the lateral position of the cells was determined by an equilibrium between two main forces, i.e. the inertial lift force F_{L} and the Dean drag force F_{D}. Due to the highly size-dependent nature of the inertial lift force F_{L}, larger cells are likely to encounter a larger F_{L}, which is strong enough to balance the F_{D} closer to the inner wall of the spiral channel. Therefore, cardiac-associated cells can be directed and concentrated to one or more outlets when the cells proceed along the spiral passageway and eventually reach the branches of the 5 separated outlets.

### Validation of the Spiral Microfluidic Device with Cardiomyocytes Spiked in Human Blood

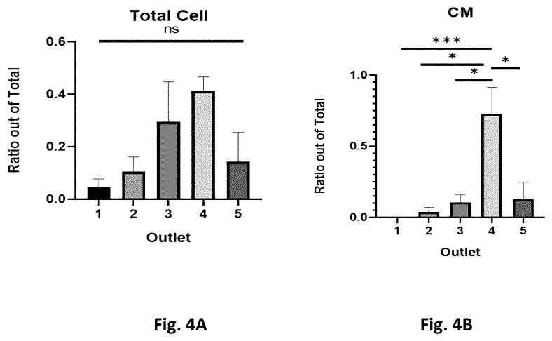

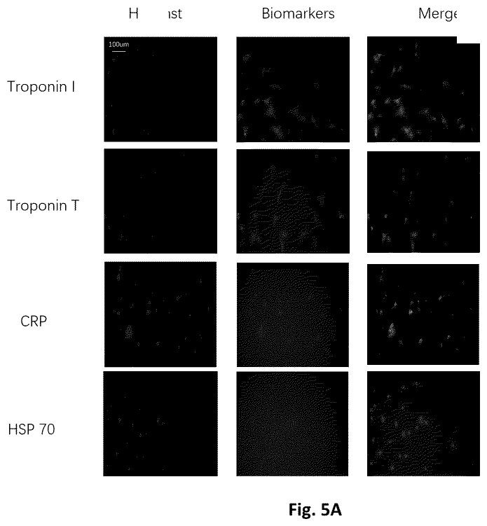

Rat cardiomyocytes were spiked onto 1 ml of whole human blood to demonstrate the clinical potential of the spiral microfluidic device for isolating cardiac-associated cells by using a small amount of blood. The components in a whole blood sample include red blood cells, platelets, leukocytes, and neutrophils, among which the cardiac-associated cells may only take up a very small proportion thereof. Particularly, red blood cells in the whole blood were first lysed using a red blood cell lysis buffer to remove signal distractions for a more sensitive test result. All the remaining nucleated cells were then loaded into the device for sorting. Results of the separation are presented in FIGS. 4A and 4B. Particularly, while all the cells were stained by Hoechst (fluorescence in blue color), only the cardiac-associated cells were stained by Calcein (fluorescence in green color). FIG. 4A shows the distribution of all of the cells counted based on Hoechst staining, in which the difference in counts from each output is statistically insignificant. FIG. 4B shows the distribution of cardiomyocytes counted based on Calcein staining, in which outlet 4 demonstrates a statistically significant difference compared to the other outputs. The results show that the majority, i.e. about 70% of the cardiomyocytes, were concentrated in outlet 4, demonstrating the efficacy of the spiral microfluidic device in isolating cardiac cells from a patient's blood sample.

### Validation of the Immunofluorescence on Cardio Fibroblast and Fibrosis-like Damage On Cardiomyocytes

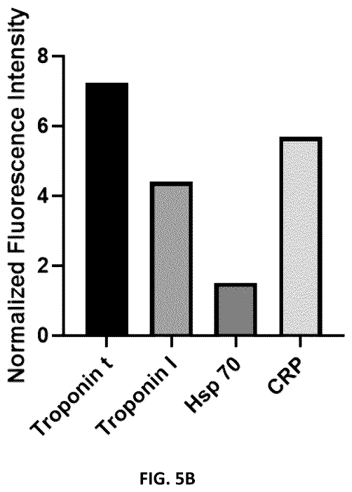

Rat cardiomyocytes were isolated from rat pups, and immunofluorescence was used to study the expression of different cell biomarkers, including C-reactive protein (CRP), heat shock protein 70 (HSP 70), Troponin I and Troponin T. The results are shown in FIGS. 5A and 5B reveal that about 14-22% of the cardiomyocytes are considered positive from these biomarkers. Particularly, amount the four tested biomarkers, Troponin T has demonstrated the most intense fluorescent signal, which agrees with reports from previous studies (Quyyumi, A. A. and A. S. Tahhan, High-Sensitivity Troponin and Coronary Artery Disease Severity: A Bridge Too Far? 2019, American College of Cardiology Foundation Washington DC. P. 1056-1057; Jin, J.-P., Evolution, regulation, and function of N-terminal variable region of troponin T: Modulation of muscle contractility and beyond. International review of cell and molecular biology, 2016. 321: p. 1-28). Interestingly, CRP, which has been reported to be expressed by the liver, was found to show a similar expression level compared to Troponin I. This demonstrates CRP may also serve as a potential biomarker for detecting CVDs from cardiomyocytes from blood samples.

### Clinical Validation of the Spiral Microfluid Device with Liquid Biopsy from Patient

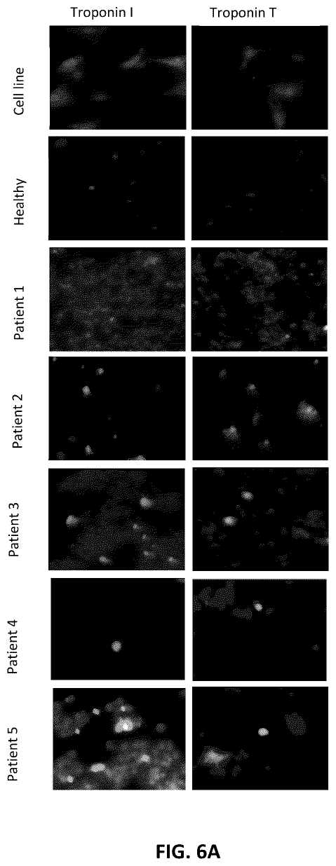

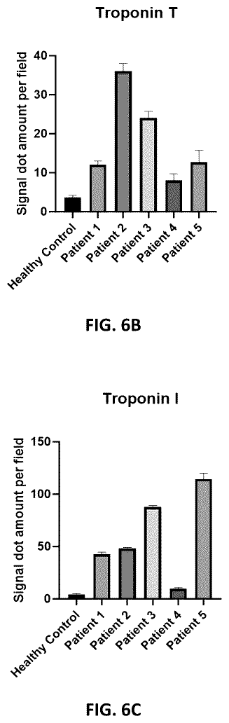

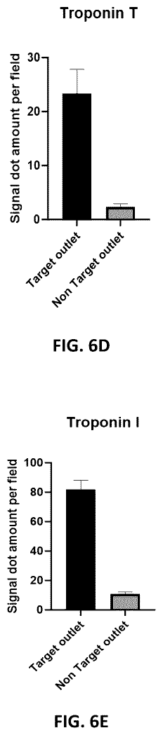

Clinical samples were collected from various subjects, including healthy individuals (Healthy Individual), patients who are feeling unwell but with no CVDs (Patient Negative), and CVD patients (Patient Positive), to investigate the ability of the spiral microfluid device of the present invention to detect CVDs in patients from their blood sample. Blood from each individual was drawn and treated with red blood cells lysis buffer before being injected into the microfluidic device. The cells collected at outlets 3 and 4 were then stained with Troponin T and Troponin I antibodies (fluorescence in green color) and Hoechst (fluorescence in blue color), respectively. Primary cardiomyocytes (Cardiomyocytes) were also included in the experiment as a positive control. Results from the immunofluorescence experiment are shown in FIGS. 6A to 6E. The signal count can be used to indicate the amount of CVDs-related cells in the collected blood sample.

The CVD detection results are shown in FIG. 6A to 6C. As shown in FIG. 6A, the sample from CVD patients has demonstrated a significantly higher signal dot amount per field compare to the healthy control. For Patient Samples 1 and 4, it appears that the samples formed agglomerates during fixation of the immunofluorescence process, which may lead to partial loss of the signal. Among the patients, Patient 4 has shown the lowest Troponin T level (i.e. about 17 ng/L) compared to others (i.e. about 200+ng/L), which may explain the lower signal strength detected.

In one trial, the cells from a non-target outlet were also collected and stained with Troponin T and I antibodies and Hoechst in order to compare with results from a target outlet. Results are shown in FIG. 6D to 6E. The signal strength of both Troponin T and Troponin I of the target outlet are almost ten times higher than that of the non-target outlet, showing the device's ability to concentrate target cells.

Notably, the Hoechst staining shows that not all of the signal dots are nucleated. In fact, most of the signal dots have no nucleus. This can be explained by the cytoplasmic fragments of the damaged cardiomyocytes which exist originally in the blood or being produced during process of the assay.

### Discussions

The present invention provides a label-free, microfluidic-based technique for the early detection and long-term monitoring of cardiovascular diseases (CVDs). The spiral microfluidic device can isolate cardiac-associated cells from a patient's blood sample, achieving analysis in a significantly shorter period when compared with traditional essays while maintaining a sufficiently high detection sensitivity. The comparison between the spiral microfluidic device of the present invention and the traditional biomarker assays is shown in Table.1.

TABLE 1
Comparison between traditional immunoassays and the
spiral microfluidic device of the present invention.

| | Traditional | Spiral Microfluidic |
|---|---|---|
| | Immunoassays | Device |
| Costs | High | Relatively low |
| Processing time | 6-10 hours | 2 hours |
| Instrument Requirements | Laboratory | Portable |
| Sensitivity | As low as 1 fmol | A few cells in 1 ml |
| | | of sample |

The present invention allows a rapid and effective detection technique that is easily adaptable in clinical settings. At the same time, the low costs and portability of the present invention enable long-term monitoring of CVDs to become practical. The technology of the present invention further allows clinicians to identify patients at risk at an early stage and thus, be able to intervene with treatment quickly to reduce mortality in CVDs patients.

While the invention has been illustrated and described in detail in the drawings and foregoing description, the same is to be considered as illustrative and not restrictive in character, it being understood that only exemplary embodiments have been shown and described and do not limit the scope of the invention in any manner. It can be appreciated that any of the features described herein may be used with any embodiment. The illustrative embodiments are not exclusive of each other or other embodiments not recited herein. Accordingly, the invention also provides embodiments that comprise combinations of one or more of the illustrative embodiments described above. Modifications and variations of the invention as herein set forth can be made without departing from the spirit and scope thereof. Therefore, only such limitations should be imposed as indicated by the appended claims.

In the claims hereof, any element expressed as a means for performing a specified function is intended to encompass any way of performing that function. The invention as defined by such claims resides in the fact that the functionalities provided by the various recited means are combined and brought together in the manner the claims call for. It is thus regarded that any means that can provide those functionalities are equivalent to those shown herein.

In the claims which follow and in the preceding description of the invention, except where the context requires otherwise due to express language or necessary implication, the word “comprise” or variations such as “comprises” or “comprising” is used in an inclusive sense, i.e., to specify the presence of the stated features but not to preclude the presence or addition of further features in various embodiments of the invention.

It is to be understood that, if any prior art publication is referred to herein, such reference does not constitute an admission that the publication forms a part of the common general knowledge in the art.

1. A method of diagnosing a cardiovascular disease (CVD), comprising the steps of:
obtaining a sample fluid from a subject;
separating components of the sample fluid into a plurality of fluid fractions;
collecting one or more selected fluid fractions comprising one or more separated components of the sample fluid, wherein the one or more separated components comprise cardiac cells; and
detecting one or more cardiovascular disease-associated biomarkers from the cardiac cells from the selected fluid fractions, thereby determining one or more related cardiovascular diseases in the subject.

2. The method according to claim 1, wherein the sample fluid is selected from the group consisting of whole blood, plasma, serum, urine or a combination thereof.

3. The method according to claim 1, further comprising the step of treating the sample fluid prior to the separating step; wherein the step of treating the sample fluid comprises lysing blood cells from the sample fluid.

4. The method according to claim 1, wherein the step of separating components of the sample fluid into a plurality of fluid fractions is conducted by using a microfluidic device; wherein the microfluidic device comprises at least a curvilinear fluid passageway connecting, at two distal ends, at least one inlet where the sample fluid is loaded, and a plurality of outlets where the corresponding plurality of fluid fractions are collected.

5. The method according to claim 4, wherein the at least one curvilinear fluid passageway of the microfluidic device comprises a spirally arranged channel forming about two to fifteen turns of substantially concentric spiral loops.

6. The method according to claim 5, wherein the inlet is arranged at a center of the substantially concentric spiral loops.

7. The method according to claim 4, wherein the microfluid device further comprises a widened channel portion connecting the curvilinear fluid passageway and the plurality of outlets.

8. The method according to claim 4, wherein the plurality of outlets comprise about two to ten outlets.

9. The method according to claim 4, wherein the curvilinear fluid passageway comprises a substantially rectangular cross-section.

10. The method according to claim 9, wherein the substantially rectangular cross-section of the curvilinear fluid passageway is of about 500 μm in width and about 200 μm in height; and wherein each of the plurality of outlets is of a cross-sectional dimension of about 300 μm in width and about 200 μm in height.

11. The method according to claim 4, wherein the step of separating components of the sample fluid into a plurality of fluid fractions is conducted by passing the sample fluid at the microfluidic device at a flow rate of about 1 ml/min to about 2 ml/min.

12. The method according to claim 4, wherein the step of separating components of the sample fluid into a plurality of fluid fractions comprises separating and focusing the components of the sample fluid based on one or more properties selected from a group consisting of size, mass, shape, surface charges, density and deformability of the components.

13. The method according to claim 4, wherein the step of separating components of the sample fluid into a plurality of fluid fractions is based on inertial migration of the components.

14. The method according to claim 1, wherein the step of detecting one or more cardiovascular disease-associated biomarkers from the cardiac cells from the selected fluid fractions comprises detecting expression of one or more cardiovascular disease-associated biomarkers from the cardiac cells from the selected fluid fractions; wherein the detecting expression of one or more cardiovascular disease-associated biomarkers from the cardiac cells from the selected fluid fractions comprises detecting immunofluorescence from the one or more cardiovascular disease-associated biomarkers from the cardiac cells separated.

15. The method according to claim 1, wherein the one or more cardiovascular disease-associated biomarkers are selected from the group consisting of troponin I, troponin T, heat shock protein 70 (HSP70), C-reactive protein (CRP), cholesterol, fibrinogen, fibrin degradation product (FDP), fetuin-A, microRNA-133a, microRNA-146a, circulating stem cells, or a combination thereof.

16. The method according to claim 1, wherein the one or more cardiovascular disease-associated biomarkers comprise physical biomarkers.

17. The method according to claim 1, wherein the cardiac cells comprise cardiomyocytes.

18. A device for separating components of a sample fluid obtained from a subject for diagnosing a cardiovascular disease (CVD) of the subject, comprising:
at least one curvilinear fluid passageway connecting, at two distal ends, at least one inlet where the sample fluid is loaded, and a plurality of outlets where a plurality of fluid fractions carrying separated components of the sample fluid are collected; wherein the separated components at one or more selected fluid fractions comprise cardiac cells.

19. A method of separating and focusing cardiac cells from a whole blood sample obtained from a subject for diagnosing a cardiovascular disease (CVD) of the subject, comprising the steps of:
treating the whole blood sample to prepare a sample fluid;
introducing the sample fluid to the device according to claim 18;
separating components of the sample fluid into a plurality of fluid fractions; and
collecting one or more selected fluid fractions comprising one or more separated cell components from the sample fluid, wherein the one or more separated cell components comprise cardiac cells.

20. The method according to claim 19, further comprising the step of detecting expression of one or more cardiovascular disease-associated biomarkers from the cardiac cells from the one or more selected fluid fractions.
